(19) United States
(12) Patent Application Publication
(10) Pub. No.: US 2013/0281503 A1
(43) Pub. Date: Oct. 24, 2013

(54) MODULATION OF RESPONSE REGULATORS BY IMIDAZOLE DERIVATIVES

(71) Applicant: North Carolina State University, (US)

(72) Inventors: Christian Melander, Raleigh, NC (US); John Cavanagh, Cary, NC (US); Richele Thompson, Orlando, FL (US)

(73) Assignee: North Carolina State University, Raleigh, NC (US)

(21) Appl. No.: 13/871,259

(22) Filed: Apr. 26, 2013

Related U.S. Application Data
(63) Continuation of application No. PCT/US2011/058277, filed on Oct. 28, 2011.
(60) Provisional application No. 61/408,378, filed on Oct. 29, 2010.

(51) Int. Cl. G01N 33/566; A01N 43/90

(52) U.S. Cl. CPC G01N 33/566; A01N 43/90
USPC 514/387; 435/7.32; 548/255

(57) ABSTRACT

The present disclosure provides methods and compositions useful for screening agents for activity in modulating response regulator signaling activity, which is, in turn, useful for determining whether these agents modulate biofilm formation or lowers the minimum inhibitory concentration (MIC) of an antibiotic, useful in determining or selecting optimum agents and/or agent dosages in modulating a biofilm of interest or lowering the minimum inhibitory concentration (MIC) of an antibiotic, and useful as a research tool for studying response regulators.

### RELATED APPLICATIONS

This application claims the benefit of U.S. Provisional Patent Application Ser. No. 61/408,378, filed Oct. 29, 2010, the disclosure of which is incorporated by reference herein in its entirety.

### FIELD

The present disclosure concerns modulation of response regulators by agents such as imidazole derivative compounds, detection thereof and compositions useful for the same.

### BACKGROUND

Numerous two component signal transduction systems (TCSTS) have been identified in bacteria, which are involved in the bacteria's ability to monitor its surroundings and adapt to changes in its environment. Response regulators are components of the TCSTS. These proteins are phosphorylated by histidine kinases, and once phosphorylated effect the response, often through a DNA binding domain becoming activated.

Among the processes regulated by TCSTS are production of virulence factors, motility, antibiotic resistance and cell replication. Inhibitors of TCSTS proteins could prevent the bacteria and other organisms having such systems from establishing and maintaining infection of the host.

There is a need for agents that effectively target and modulate TCSTS members. Such agents may be useful as anti-infectives or used as agents for research purposes to aid in determining their roles in pathogenesis of infection, dysfunction and disease.

### SUMMARY

The present disclosure provides methods and compositions useful for screening agents for activity in modulating response regulator signaling activity, which is, in turn, useful for determining whether these agents modulate biofilm formation, modulate of microorganism growth or lowers the minimum inhibitory concentration (MIC) of an antibiotic, useful in determining or selecting optimum agents and/or agent dosages in modulating a biofilm of interest, modulating of microorganism growth or lowering the minimum inhibitory concentration (MIC) of an antibiotic, useful as a research tool for studying response regulators, etc.

In some embodiments, methods are provided for screening an agent (e.g., a compound having an imidazole moiety, such as a compound of Formula (X)(I)(a), Formula (I), Formula (I)(a)(1), Formula (II), Formula (IV), Formula (V), or Formula (VI) as described herein, or a pharmaceutically acceptable salt or prodrug thereof), for inhibition of biofilm formation, modulation of microorganism growth or lowering the minimum inhibitory concentration (MIC) of an antibiotic, including (a) contacting (in vitro or in vivo) the agent to a response regulator, and then (b) detecting the presence or absence of binding of the compound to the response regulator, the presence of binding indicating that the agent has activity in inhibition of biofilm formation, modulation of microorganism growth or lowering the minimum inhibitory concentration (MIC) of an antibiotic.

In some embodiments, methods of selecting an agent are provided (e.g., a compound having an imidazole moiety, such as a compound of Formula (X)(I)(a), Formula (I), Formula (I)(a)(1), Formula (II), Formula (IV), Formula (V), or Formula (VI) as described herein, or a pharmaceutically acceptable salt or prodrug thereof), for activity in treating, reducing or removing a biofilm of interest, modulating of microorganism growth or lowering the minimum inhibitory concentration (MIC) of an antibiotic, including: (a) providing a sample (e.g., a lysate sample) of the biofilm of interest (having known/predetermined and/or unknown/not predetermined microorganism species therein), the sample having a response regulator therein, or providing an isolated response regulator from a known/predetermined microorganism species, and (b) assaying for binding of the agent to the response regulator, which binding indicates the agent has activity in treating, reducing or removing the biofilm of interest, modulating microorganism growth or lowering the minimum inhibitory concentration (MIC) of an antibiotic.

Also provided are compositions (e.g., aqueous compositions) having: (a) an isolated, purified or substantially purified response regulator, and (b) an agent, e.g., a compound having an imidazole moiety, such as a compound of Formula (X)(I)(a), Formula (I), Formula (I)(a)(1), Formula (II), Formula (IV), Formula (V), or Formula (VI) as described herein, or a pharmaceutically acceptable salt or prodrug thereof.

In some embodiments, provided are kits having: (a) an isolated, purified or substantially purified response regulator, and (b) an agent, e.g., a compound having an imidazole moiety, such as a compound of Formula (X)(I)(a), Formula (I), Formula (I)(a)(1), Formula (II), Formula (IV), Formula (V), or Formula (VI) as described herein, or a pharmaceutically acceptable salt or prodrug thereof, (c) a container, and optionally, (d) instructions for use.

Also provided are agents (e.g., a compound having an imidazole moiety, such as a compound of Formula (X)(I)(a), Formula (I), Formula (I)(a)(1), Formula (II), Formula (IV), Formula (V), or Formula (VI) as described herein, or a pharmaceutically acceptable salt or prodrug thereof) coupled to a detectable group (e.g., covalently coupled). In some embodiments, the detectable group is a radiolabel, a gold bead, a chemiluminescence label, a ligand (e.g., biotin, digoxin), a fluorescence label (e.g., rhodamine, phycoerythrin, fluorescein), a fluorescent protein (e.g., green fluorescent protein or one of its many modified forms), a nucleic acid, or an energy absorbing/emitting agent.

In some embodiments of the methods and compositions provided herein, the response regulator is an OmpR, NarL, NtrC, BfmR, GacA, LytTR, AraC, Spo0A, F is, YcbB, RpoE, MerR, GGDEF, EAL, HD-GYP, CheB, CheC, PP2C, HisK, ANTAR, CsrA, PAS, GAF, TPR, CAP_ED, HPt, PhoB, CheY, a portion thereof (e.g., the N-terminal portion thereof), or an analog or homolog of the same. For example, the response regulator may be an A. baumannii BfmR protein, a P. aeuriginosa GacA protein, or an E. coli OmpR protein, a portion thereof (e.g., an N-terminal portion thereof), or an analog or homolog of the same.

In some embodiments of the methods and compositions provided herein, the agent and/or response regulator is coupled to a detectable group, e.g., a radiolabel, a gold bead, a chemiluminescence label, a ligand (e.g., biotin, digoxin), a fluorescence label (e.g., rhodamine, phycoerythrin, fluorescein), a fluorescent protein (e.g., green fluorescent protein or one of its many modified forms), a nucleic acid, or an energy absorbing/emitting agent.

Also provided are methods of inhibiting biofilm formation of a cellular species capable of such formation (e.g., bacteria of the genus Acinetobacter, Pseudomonas, or Vibrio), including binding of a response regulator (e.g., OmpR, BfmR, or GacA) of the cellular species with an exogenous agent (e.g., a compound having an imidazole moiety, such as a compound of Formula (X)(I)(a), Formula (I), Formula (I)(a)(1), Formula (II), Formula (IV), Formula (v), or Formula (VI) as described herein, or a pharmaceutically acceptable salt or prodrug thereof) provided in an amount effective to modulate biofilm-mediating communication of the cellular species, to thereby inhibit biofilm formation.

Further provided are methods of modulating biofilm activity of a biofilm-producing cellular species (e.g., bacteria of the genus Acinetobacter, Pseudomonas, or Vibrio), including binding a surface bound response regulator (e.g., BfmR) of cells of the cellular species with an exogenous agent (e.g., a compound having an imidazole moiety, such as a compound of Formula (X)(I)(a), Formula (I), Formula (I)(a)(1), Formula (II), Formula (IV), Formula (v), or Formula (VI) as described herein, or a pharmaceutically acceptable salt or prodrug thereof) provided in an amount effective to at least partially suppress biofilm-mediating response regulator/histidine kinase communication of the cells.

Provided in some embodiments are methods of disrupting quorum sensing of a cellular species capable of biofilm production (e.g., bacteria of the genus Acinetobacter, Pseudomonas, or Vibrio), including binding a surface bound response regulator (e.g., BfmR) of cells of the cellular species with an exogenous agent (e.g., a compound having an imidazole moiety, such as a compound of Formula (X)(I)(a), Formula (I), Formula (I)(a)(1), Formula (II), Formula (IV), Formula (V), or Formula (VI) as described herein, or a pharmaceutically acceptable salt or prodrug thereof) provided in an amount effective to at least partially suppress biofilm-mediating response regulator/histidine kinase communication of the cells.

Also provided are methods of inhibiting antibiotic resistance of a cellular species capable of biofilm production (e.g., bacteria of the genus Acinetobacter, Pseudomonas, or Vibrio), including binding a surface bound response regulator (e.g., BfmR) of cells of the cellular species with an exogenous agent (e.g., a compound having an imidazole moiety, such as a compound of Formula (X)(I)(a), Formula (I), Formula (I)(a)(1), Formula (II), Formula (IV), Formula (v), or Formula (VI) as described herein, or a pharmaceutically acceptable salt or prodrug thereof) provided in an amount effective to at least partially suppress biofilm-mediating response regulator/histidine kinase communication of the cells, to thereby inhibit antibiotic resistance.

Further provided is the use of an agent such as a compound having an imidazole moiety in the foregoing methods.

### BRIEF DESCRIPTION OF THE DRAWINGS

FIG. 1 is a photograph of an SDS-PAGE gel showing binding of a biotoin-coupled imidazole derivative compound to a BfmR response regulator in A. baumannii lysate. From Left to Right: Lane 1 Molecular Weight Marker; Lane 2 BfmR; Lanes 3-5 washes; Lanes 6-7 elution; Lane 8 is an elution (but is obscured by the lysate bleed through in lane 9); (Lane 10 and 11 also have bleed through); Lane 12 is a wash of the beads; Lane 13-14 elution of A. Baumannii lysate from the compound; Lane 15 blank.

FIG. 2 is a photograph of a western blot confirming that the protein bound to the biotin-coupled imidazole derivative compound is A. baumannii BfmR with polyclonal antibodies specific to BfmR. From Left to Right: Lane 1: A. baumannii lysate; Lane 2-4: Elution of protein from A. baumannii lysate that bound to compound; Lane 5: Molecular weight marker Lane 6: empty; Lane 7: BfmR conc. sample; Lane 8-10 Elution of BfmR bound to compound

FIG. 3 presents an amino acid sequence alignment between BfmR from A. baumannii, OmpR from E. coli, and a response regulator (RR) protein from X. campestris. Conserved structural regions are indicated. A: N-terminal regulatory domain that gets phosphrylated in a conserved aspartic acid binding pocket. B: Flexible loop/linker region between N- and C-terminal domains. C: C-terminal DNA binding domain.

FIGS. 4A-4B are pictures of computational docking of two imidazole derivative compounds with two response regulators. In both instances, the N-terminal domain is preferentially targeted. 4A: imidazole derivative compound bound to A. baumannii BfmR. 4B: different imidazole derivative compound bound to P. aeuriginosa GacA.

### BRIEF DESCRIPTION OF THE SEQUENCE LISTING

The present application includes a sequence listing, which is incorporated herein by reference in its entirety.

SEQ ID NO:1 presents the amino acid sequence of A. baumannii BfmR, accession no. AAX40744.1.

SEQ ID NO:2 presents the amino acid sequence of E. coli OmpR, accession no. NP_{—}417864.1.

SEQ ID NO:3 presents the amino acid sequence of X. campestris RR protein, accession no. ZP_{—}06484176.1.

SEQ ID NO:4 presents the amino acid sequence of P. aeuriginosa GacA, accession no. AAA68948.1

SEQ ID NO:5 presents the amino acid sequence of P. aeuriginosa BfmR, accession no. NP 252790.1.

### DETAILED DESCRIPTION

The present disclosure provides methods and compositions useful for screening agents that have activity in modulating response regulator signaling activity, which is, in turn, useful for determining whether these agents modulate biofilm formation, useful in determining or selecting optimum agents and/or agent dosages in modulating a biofilm of interest or lowering the minimum inhibitory concentration (MIC) of an antibiotic, useful as a research tool for studying response regulators, etc. The screening of response regulators and/or agents (e.g., imidazole derivative compounds) as described herein can be carried out in vitro or in vivo by any suitable method, numerous variations of which will be apparent to those skilled in the art.

All patent references referred to herein are incorporated by reference in their entireties.

In some embodiments, screening is carried out in vitro by performing an assay which detects the binding of an agent (e.g., a compound containing an imidazole moiety) to one or more response regulators. Assays used to detect the binding of an agent to one or more response regulators are known in the art and include, but are not limited to, pull down assays, western blot assays, enzyme-linked immunosorbent assays (ELISA), etc.

The response regulators according to some embodiments are isolated. “Isolated” as used herein means that they are removed through human manipulation from their natural environment in the cell and/or extracellular space. In some embodiments, the response regulators are provided in a purified or substantially purified form, such that all or substantially all of the cellular components (e.g., other cellular proteins) are not present. In other embodiments, the response regulator proteins are provided as part of a cell lysate or a portion thereof.

In some embodiments, a sample of a biofilm of interest (comprising known and/or unknown bacteria) can be provided, which sample may be tested for the binding of an agent to, e.g., one or more proteins contained in a lysate (e.g., response regulator proteins), which binding indicates biofilm modulating activity (e.g., biofilm inhibition) of said biofilm of interest. Such testing according to some embodiments may be useful to determine the effectiveness of the agent in modulating the biofilm of interest (e.g., an animal (e.g., human) or plant infection) or lowering the minimum inhibitory concentration (MIC) of an antibiotic.

Response regulators and/or agents may in some embodiments be coupled to a detectable group as appropriate for the assay being performed. “Detectable groups” as used herein include, but are not limited to, radiolabeling with a radionuclide (e.g., ³⁵S, ¹²⁵I, ³²P, ³H, ¹⁴C, ¹³¹I), enzyme labels (e.g., horseradish peroxidase, alkaline phosphatase), gold beads, chemiluminescence labels, ligands (e.g., biotin, digoxin) and/or fluorescence labels (e.g., rhodamine, phycoerythrin, fluorescein), a fluorescent protein including, but not limited to, green fluorescent protein or one of its many modified forms, a nucleic acid segment in accordance with known techniques, and energy absorbing and energy emitting agents.

“Radionuclide” as described herein may be any radionuclide suitable for delivering a therapeutic dosage of radiation to a tumor or cancer cell, including, but not limited to, ²²⁵Ac, ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰Y, ²¹²Bi, ²¹³Bi, ¹¹⁹Sb, ¹⁹⁷Hg, ⁹⁷Ru, ¹⁰⁰Pd, ^{101m}Rh, and ²¹²Pb.

“Energy absorbing and energy emitting agent” as used herein includes, but is not limited to, diagnostic agents, contrast agents, iodinated agents, radiopharmaceuticals, fluorescent compounds and fluorescent compounds coencapsulated with a quencher, agents containing MRS/MRI sensitive nuclides, genetic material encoding contrast agents, and energy absorbing and heat emitting nanomaterials including, but not limited to, single-walled nanotubes and gold nanocages. Some examples of contrast agents include, but are not limited to, metal chelates, polychelates, multinuclear cluster complexes (U.S. Pat. No. 5,804,161), halogenated xanthene or a functional derivative of a halogenated xanthene (U.S. Pat. No. 6,986,740), gadolinium-diethylenetriaminepentaacetic acid (gadopentetate dimeglumine, GdDTPA; Magnavist), gadoteridol (ProHance), gadodiamide, gadoterate meglumine (Gd-DOTA), gadobenate dimeglumine (Gd-BOPTA/Dimeg; MultiHance), mangafodipir trisodium (Mn-DPDP), ferumoxides, paramagnetic analogue of doxorubicin, and ruboxyl (Rb). Some examples of iodinated agents include, but are not limited to, diatrizoate(3,5-di(acetamido)-2,4,6-triiodobenzoic acid), iodipamide (3,3′-adipoyl-diimino-di(2,4,6-triiodobenzoic acid), acetrizoate[3-acetylamino-2,4,6-triiodobenzoic acid], aminotrizoate[3-amino-2,4,6-triiodobenzoic acid]), and iomeprol. Examples of radiopharmaceuticals include, but are not limited to, fluorine-18 fluorodeoxyglucose ([18F]FDG), Tc-99m Depreotide, carbon-11 hydroxyephedrine (HED), [18F]setoperone, [methyl-11C]thymidine, 99 mTc-hexamethyl propyleneamine oxime (HMPAO), 99 mTc-L, L-ethylcysteinate dimer (ECD), 99 mTc-sestamibi, thallium 201, I-131metaiodobenzylguanidine (MIBG), 123I—N-isopropyl-p-iodoamphetamine (IMP), 99 mTc-hexakis-2-methoxyisobutylisonitrile (MIBI), 99 mTc-tetrofosmin. Examples of agents containing MRS/MRI sensitive nuclides include, but are not limited to, perfluorocarbons and fluorodeoxyglucose. Examples of genetic material encoding contrast agents include, but are not limited to, paramagnetic reporter genes such as ferredoxin; paramagnetic tag(s) on liposomal lipids such as paramagnetic chelating groups added to PEG; detectable probes; and luciferin/luciferase reporter system.

“Biofilm” or “biofilms” refer to communities of microorganisms that are attached to a substrate. The microorganisms often excrete a protective and adhesive matrix of polymeric compounds. They often have structural heterogeneity, genetic diversity, and complex community interactions. “Biofilm inhibiting” refers to inhibition or slowing of the establishment or growth of a biofilm, or decrease in the amount of organisms that attach and/or grow upon a substrate, up to and including the complete removal of the biofilm. As used herein, a “substrate” can include any living or nonliving structure. “Planktonic” bacteria are bacteria that are free-swimming in a fluid, as opposed to attached to a surface (such as in a biofilm).

The “minimum inhibitory concentration” or “MIC” of an antibiotic, as known in the art, is the lowest concentration of an antibiotic that will inhibit the visible growth of a microorganism after overnight incubation (e.g., 16 hours). MICs can be measured by methods well known in the art, such as agar or broth dilution methods following the guidelines of CLSI, BSAC or EUCAST, commercially available E-test strips, Oxoid MICEvaluator methods, etc. See, e.g., CLSI (2009) Performance standards for antimicrobial susceptibility testing; 19th informational supplement, p. 152, Clinical and Laboroaty Standards Institute, Wayne, Pa.; Rogers et al., Synergistic effects between conventional antibiotics and 2-aminoimidazole-derived antibiofilm agents, Antimicrobial Agents and Chemotherapy, May 2010, p. 2112-2118.

Common antibiotics include aminoglycosides, carbacephems (e.g., loracarbef), carbapenems, cephalosporins, glycopeptides (e.g., teicoplanin and vancomycin), macrolides, monobactams (e.g., aztreonam) penicillins, polypeptides (e.g., bacitracin, colistin, polymyxin B), quinolones, sulfonamides, tetracyclines, etc. Antibiotics treat infections by either killing or preventing the growth of microorganisms. Many act to inhibit cell wall synthesis or other vital protein synthesis of the microorganisms.

Aminoglycosides are commonly used to treat infections caused by Gram-negative bacteria such as Escherichia coli and Klebsiella, particularly Pseudomonas aeroginosa. Examples of aminoglycosides include, but are not limited to amikacin, gentamicin, kanamycin, neomycin, netilmicin, streptomycin, tobramycin, and paromomycin.

Carbapenems are broad-specrum antibiotics, and include, but are not limited to, ertapenem, doripenem, imipenem/cilstatin, and meropenem.

Cephalosporins include, but are not limited to, cefadroxil, cefazolin, cefalotin (cefalothin), cefalexin, cefaclor, cefamandole, cefoxitin, cefprozil, loracarbef, cefuroxime, cefixime, cefdinir, cefditoren, cefoperazone, cefotaxime, cefpodoxime, ceftazidime, ceftibuten, ceftizoxime, ceftriaxone, cefepime, cefpirome, and ceftobiprole.

Macrolides include, but are not limited to, azithromycin, clarithromycin, dirithromycin, erythromycin, roxithromycin, troleandomycin, telithromycin and spectinomycin.

Penicillins include, but are not limited to, amoxicillin, ampicillin, azlocillin, bacampicillin, carbenicillin, cloxacillin, dicloxacillin, flucloxacillin, mezlocillin, meticillin, nafcillin, oxacillin, penicillin, piperacillin and ticarcillin.

Quinolones include, but are not limited to, ciprofloxacin, enoxacin, gatifloxacin, gemifloxacin, levofloxacin, lomefloxacin, moxifloxacin, norfloxacin, ofloxacin and trovafloxacin.

Sulfonamides include, but are not limited to, mafenide, prontosil, sulfacetamide, sulfamethizole, sulfanilamide, sulfasalazine, sulfisoxazole, trimethoprim, and co-trimoxazole (trimethoprim-sulfamethoxazole).

Tetracyclines include, but are not limited to, demeclocycline, doxycycline, minocycline, oxytetracycline and tetracycline.

Other antibiotics include arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin (rifampicin), tinidazole, etc.

### A. Cells and Response Regulators

“Cells” used in the assays according to some embodiments may include any kind of cell which expresses or is capable of expressing (i.e., contains a gene encoding) one or more response regulator proteins, and includes microorganisms (e.g., bacteria, fungal cells, etc.). Cells may be provided as all or substantially all from a particular microorganism of interest, or may be a mixture of cells containing more than one species of microorganisms (e.g., collected from a biofilm of interest, such as from a substrate to be treated).

“Response regulator” proteins are known and found in bacteria (e.g., Gram-negative and Gram-positive), archaea, fungi, and some eukaryotic cells. The response regulator proteins are generally characterized by a conserved N-terminal sensor/receptor domain and a conserved C-terminal transcription regulatory DNA-binding domain. Response regulators include, but are not limited to, those in the OmpR, NarL, and NtrC families, analogs or homologs thereof, etc. See, e.g., Glaperin, “Structural Classification of Bacterial Response Regulators: Diversity of Output Domains and Domain Combinations,” J. Bacteriology, June 2006, p. 4169-4182. Additional exemplary response regulators include, but are not limited to, BfmR, GacA, LytTR, AraC, Spo0A, F is, YcbB, RpoE, MerR, GGDEF, EAL, HD-GYP, CheB, CheC, PP2C, HisK, ANTAR, CsrA, PAS, GAF, TPR, CAP_ED, HPt, PhoB, CheY, and analogs or homologs thereof. Response regulator proteins may in some embodiments be from Gram-negative or Gram-positive bacteria. For example, response regulators may be of the Gram-negative genera Escherichia (e.g., E. coli OmpR), Salmonella, Vibrio, Helicobacter, Pseudomonas (e.g., P. aeuriginosa GacA), Bordetella, Vibrio, Haemophilus, Halomonas, and/or Acinetobacter (e.g., A. baumannii BmfR).

Response regulator proteins, homologs or portions thereof (e.g., including a specific domain thereof), or proteins having substantial sequence similarity to the same, may be used in the assays as provided herein. The proteins may be obtained by purification from the natural host, by recombinant protein expression, or by any other means commonly employed in the art.

The phrase “substantial sequence similarity” means that amino acid sequences which have slight and non-consequential sequence variations from the actual sequences disclosed and claimed herein are considered to be equivalent to the sequences of the present invention. In this regard, “slight and non-consequential sequence variations” mean that “similar” sequences (i.e., the sequences that have substantial sequence similarity with proteins discussed herein) will be functionally equivalent to the sequences disclosed and claimed in the present invention. Functionally equivalent sequences will function in substantially the same manner to produce substantially the same compositions as the amino acids/proteins discussed herein.

In some embodiments, a homolog or portion of a response regulator may be used in the assays as provided herein. The homolog or portion of a response regulator may contain all, substantially all or part of one or more specific domains of a response regulator, such as, but not limited to, a conserved N-terminal sensor/receptor domain, a conserved C-terminal transcription regulatory DNA-binding domain, a REC domain, a RNA-binding domain, an enzymatic domain, a protein- or ligand-binding domain, and any combination thereof.

For example, a protein comprising one or more domains of a response regulator (e.g., the N-terminal regulatory domain) may be attached to a solid support, and compounds screened for binding thereto in an in vitro assay.

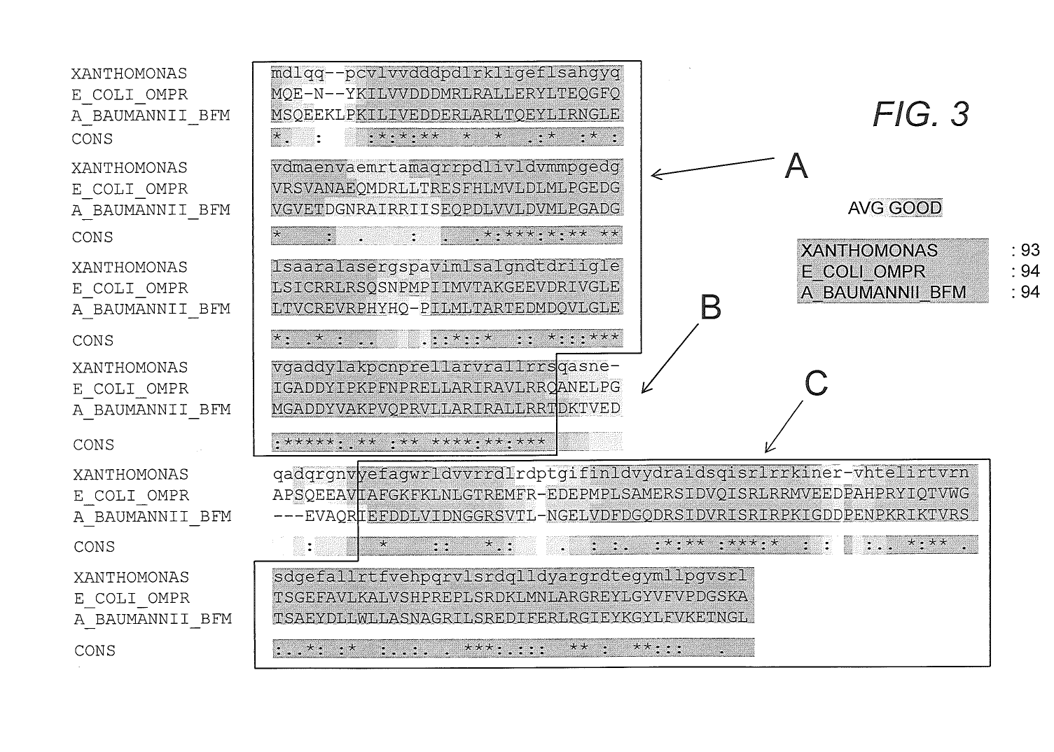

The N-terminal regulatory domain, also known as the “REC” or signal receiver domain (NCBI cd00156), has a phosphorylation site in a conserved aspartic acid binding pocket (see FIG. 3). This domain is highly conserved across species, and generally is included in the N-terminal 100, 125, or 150 amino acids of the response regulator protein. See, e.g., Volz et al., “Crystal structure of Escherichia coli CheY refined at 1.7-A resolution,” J Biol Chem 1991, 266(23):15511-9; Volz, “Structural conservation in the CheY superfamily,” Biochemistry 1993, 32(44):11741-53; Mottonen et al., “Allosteric response is both conserved and variable across three CheY orthologs,” Biophys J 2010, 99(7):2245-54.

Gram-negative bacteria are those that do not retain crystal violet dye after an alcohol wash in the Gram staining protocol. This is due to structural properties in the cell walls of the bacteria. Many genera and species of Gram-negative bacteria are pathogenic. Gram-negative bacteria include members of the phylum proteobacteria, which include genus members Escherichia, Salmonella, Vibrio, and Helicobacter. Examples of genera of biofilm-forming bacteria include, but are not limited to, Pseudomonas, Bordetella, Vibrio, Haemophilus, Halomonas, and Acinetobacter. Other genera include Klebsiella, Proteus, Neisseria, Helicobacter, Brucella, Legionella, Campylobacter, Francisella, Pasteurella, Yersinia, Bartonella, Bacteroides, Streptobacillus, Spirillum, Moraxella and Shigella. Examples of species of bacteria include Pseudomonas aeuroginosa, Bordetella pertussis, Vibrio vulnificus, Haemophilus influenzae, and Halomonas pacifica.

Gram-negative bacteria of the Acinetobacter genus belong to the phylum Gammaproteobacteria, order Pseudomonadalas, and family Moraxellaceae. Genus members include, but are not limited to, Acinetobacter bumannii, Acinetobacter haemolyticus, and Acinetobacter lwoffi. Various nosocomial infections that are especially prevalent in intensive care units implicate Acinetobacter species such as Acinetobacter baumannii and Acinetobacter lwoffi. Acinetobacter baumanni is a frequent cause of nosocomial pneumonia, and can also cause skin and wound infections and bacteremia. Acinetobacter lwoffi causes meningitis. The Acinetobacter species are resistant to many classes of antibiotics.

Examples of Gram-positive bacteria include, but are not limited to, bacteria of the genera Listeria, Staphylococcus, Streptococcus, Bacillus, Corynebacterium, Peptostreptococcus, and Clostridium. Species include, but not limited to, Listeria monocytogenes, Staphylococcus aureus, Streptococcus pyogenes, Streptococcus pneumoniae, Bacillus cereus, Bacillus anthracia, Clostridium botulinum, Clostridium perfringens, Clostridium difficile, Clostridium tetani, Corynebacterium diphtheriae, Corynebacterium ulcerans, and Peptostreptococcus anaerobius. Other bacterial genera include, but are not limited to, Actinomyces, Propionibacterium, Nocardia and Streptomyces.

Staphylococcus aureus is a common cause of nosocomial infections, often found in post-surgical wound infections. Staphylococcus aureus can also cause a variety of other infections in humans (e.g., skin infections), as well as contribute to mastitis in dairy cows. Methicillin-resistant Staphylococcus aureaus (MRSA), in particular, is especially difficult to treat due to multiple drug resistances, including penicillins and cephalosporins. MRSA has become problematic in hospital settings, particularly among the more susceptible patients with open wounds, invasive devices, weakened immune systems, etc.

Various bacteria which are known to infect plants include, but are not limited to, Xanthomonas species such as Xanthomonas campestris, Xanthomonas campestris pv. vesicatoria, Xanthomonas axonopodis pv. Glycines, and Xanthomonas axonopodis, Pseudomonas species such as Pseudomonas syringae, Pseudomonas corrugate, and Pseudomonas amygdale, Xylella species such as Xylella fastidiosa, Candidatus Liberibacter asiaticus, Erwinia species such as Erwinia amylovora, Erwinia chrysanthemi, Erwinia ananas, Erwinia herbicola and Erwinia carotovora, Agrobacterium species such as Agrobacterium tumefaciens and Agrobacterium rhizogenes, Ralstonia solanacearum, Clavibacter michiganesis, Curtobacterium flaccumfaciens,

A “fungal cell” as used herein may be any fungal cell belonging to the genera including, but not limited to, Aspergillus, Candida, Cryptococcus, Coccidioides, Tinea, Sporothrix, Blastomyces, Histoplasma, Pneumocystis and Saccharomyces. Additionally, fungal cells include, but is not limited to, Aspergillus fumigatus, Aspergillus flavus, Aspergillus niger, Aspergillus terreus, Aspergillus nidulans, Candida albicans, Coccidioides immitis, Cryptococcus neoformans, Tinea unguium, Tinea corporis, Tinea cruris, Sporothrix schenckii, Blastomyces dermatitidis, Histoplasma capsulatum, Histoplasma duboisii, and Saccharomyces cerevisiae. Further examples include, but are not limited to, Pythium spp., Fusarium spp., Rhizoctonia spp., Cercospora spp., Alternaria spp., Colletotrichum spp., Ustilago spp., Phoma spp., Gibberella spp. Penicillium spp., Glomerella spp. Diplodia spp., Curvularia spp., Sclerospora spp., Peronosclerospora spp., Cercospora spp., Puccinia spp., Ustilago spp., Phomopsis spp., Diaporthe spp., Botrytis spp., Verticillium spp., and Phytophthors spp.

### B. Modulation Agents

Agents that may be used in the methods and compositions disclosed herein include compounds having an imidazole moiety therein, including imidazole derivative compounds. Imidazole derivative compounds include those compounds described in U.S. Patent Application Publication numbers 2008/0181923, 2009/0143230, 2009/0263438, and 2009/0270475 to Melander et al.; and PCT Application Publication number WO 2010/077603 to Melander et al., the disclosures of which are hereby incorporated by reference herein in their entireties. The compounds can be prepared as detailed in these referenced application publications or in accordance with known procedures or variations thereof that will be apparent to those skilled in the art. In some embodiments, the imidazole derivative compound is coupled (e.g., covalently coupled) to a detectable group.

In some embodiments, the compounds inhibit biofilm formation, modulate microorganism growth, and/or lower the minimum inhibitory concentration (MIC) of an antibiotic by binding to one or more response regulators as taught herein. See also Rogers et al., “Chemical synthesis and biological screening of 2-aminoimidazole-based bacterial and fungal antibiofilm agents.” Chem Bio Chem, 2010, 11 (3), 396-410; Rogers et al., “Synergistic effects between conventional antibiotics and 2-aminoimidazole-derived antibiofilm agents,” Antimicrobial Agents and Chemotherapy, May 2010, p. 2112-2118.

In some embodiments, the compounds effectively disable bacteria's ability to upregulate inducible resistance genes by binding to response regulators, interfering with the bacteria's two-component system, which allows the bacteria to sense their surroundings. In this manner, the bacteria's ability to sense their environment is disabled, and gene expression of inducible resistance mechanisms is decreased or not triggered, mitigating the development or maintenance of resistance in bacteria and rendering them more responsive to antibiotics (and thus lowering their MIC). The response regulator proteins are essentially ubiquitous in the bacterial kingdom, providing a mechanistic basis for activity across Gram-positive and Gram-negative bacteria.

As will be appreciated by those of skill in the art, the compounds of the various formulas disclosed herein may contain chiral centers, e.g. asymmetric carbon atoms. The present disclosure is concerned with the use of both: (i) racemic mixtures of the active compounds, and (ii) enantiomeric forms of the active compounds. The resolution of racemates into enantiomeric forms can be done in accordance with known procedures in the art. For example, the racemate may be converted with an optically active reagent into a diastereomeric pair, and the diastereomeric pair subsequently separated into the enantiomeric forms.

Geometric isomers of double bonds and the like may also be present in the compounds, and all such stable isomers are included within the present disclosure unless otherwise specified. Also included are tautomers (e.g., tautomers of imidazole) and rotamers.

“Imidazole” refers to the commonly known structure:

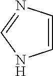

“H” refers to a hydrogen atom. “C” refers to a carbon atom. “N” refers to a nitrogen atom. “O” refers to an oxygen atom. “Halo” refers to F, Cl, Br or I. The term “hydroxy,” as used herein, refers to an —OH moiety. “Br” refers to a bromine atom. “Cl” refers to a chlorine atom. “I” refers to an iodine atom. “F” refers to a fluorine atom.

An “acyl group” is intended to mean a —C(O)—R radical, where R is a suitable substituent (for example, an acetyl group, a propionyl group, a butyroyl group, a benzoyl group, or an alkylbenzoyl group).

“Alkyl,” as used herein, refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. In some embodiments, alkyl groups as described herein are optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

The term “optionally substituted” indicates that the specified group is either unsubstituted, or substituted by one or more suitable substituents. A “substituent” is an atom or atoms substituted in place of a hydrogen atom on the parent chain or cycle of an organic molecule, for example, H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

“Alkenyl,” as used herein, refers to a straight or branched chain hydrocarbon containing from 1 or 2 to 10 or 20 or more carbons, and containing at least one carbon-carbon double bond, formed structurally, for example, by the replacement of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. In some embodiments, alkenyl groups as described herein are optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

“Alkynyl,” as used herein, refers to a straight or branched chain hydrocarbon group containing from 1 or 2 to 10 or 20 or more carbon atoms, and containing at least one carbon-carbon triple bond. Representative examples of alkynyl include, but are not limited, to acetylenyl, 1-propynyl, 2-propynyl, 3-butynyl, 2-pentynyl, 1-butynyl and the like. In some embodiments, alkynyl groups as described herein are optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

The term “cycloalkyl,” as used herein, refers to a saturated cyclic hydrocarbon group containing from 3 to 8 carbons or more. Representative examples of cycloalkyl include, cyclopropyl, cyclobutyl, cyclopentyl, cyclohexyl, cycloheptyl, and cyclooctyl. In some embodiments, cycloalkyl groups as described herein are optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

“Heterocyclo,” as used herein, refers to a monocyclic or a bicyclic ring system. Monocyclic heterocycle ring systems are exemplified by any 5 or 6 member ring containing 1, 2, 3, or 4 heteroatoms independently selected from the group consisting of: O, N, and S. The 5 member ring has from 0 to 2 double bonds, and the 6 member ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, sulfoxide, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, and the like.

“Aryl” as used herein refers to a fused ring system having one or more aromatic rings. Representative examples of aryl include, azulenyl, indanyl, indenyl, naphthyl, phenyl, tetrahydronaphthyl, and the like. The aryl groups of this disclosure can be substituted with 1, 2, 3, 4, or 5 substituents independently selected from alkenyl, alkenyloxy, alkoxy, alkoxyalkoxy, alkoxycarbonyl, alkyl, alkylcarbonyl, alkylcarbonyloxy, alkylsulfinyl, alkylsulfonyl, alkylthio, alkynyl, aryl, aryloxy, azido, arylalkoxy, arylalkyl, aryloxy, carboxy, cyano, formyl, halogen, haloalkyl, haloalkoxy, hydroxy, hydroxyalkyl, mercapto, nitro, sulfamyl, sulfo, sulfonate, —NR′R″ (wherein, R′ and R″ are independently selected from hydrogen, alkyl, alkylcarbonyl, aryl, arylalkyl and formyl), and —C(O)NR′R″ (wherein R′ and R″ are independently selected from hydrogen, alkyl, alkylcarbonyl, aryl, arylalkyl, and formyl).

“Heteroaryl” means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from the group consisting of: O, N, and S. The heteroaryl group, including each heteroatom, can be unsubstituted or substituted with from 1 to 4 suitable substituents, as chemically feasible. For example, the heteroatom S may be substituted with one or two oxo groups, which may be shown as ═O.

“Alkoxy,” as used herein, refers to an alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like. In some embodiments, alkoxy groups as described herein are optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

An “amine” or “amino” group is intended to mean the group —NH₂. “Optionally substituted” amines refers to —NH₂ groups wherein none, one or two of the hydrogens is replaced by a suitable substituent as described herein, such as alkyl, alkenyl, alkynyl, cycloalkyl, heterocyclo, aryl, heteroaryl, alkoxy, carbonyl, carboxy, etc. In some embodiments, one or two of the hydrogens are optionally substituted with independently selected, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide. Disubstituted amines may have substituents that are bridging, i.e., form a heterocyclic ring structure that includes the amine nitrogen.

An “amide” as used herein refers to an organic functional group having a carbonyl group (C═O) linked to a nitrogen atom (N), or a compound that contains this group, generally depicted as:

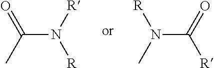

wherein, R and R′ can independently be any covalently-linked atom or atoms, for example, H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

A “thiol” or “mercapto” refers to an —SH group or to its tautomer ═S.

A “sulfone” as used herein refers to a sulfonyl functional group, generally depicted as:

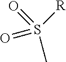

wherein, R can be any covalently-linked atom or atoms, for example, H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

A “sulfoxide” as used herein refers to a sulfinyl functional group, generally depicted as:

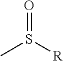

wherein, R can be any covalently-linked atom or atoms, for example, H, halohydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

“Triazole” refers to the commonly known structures:

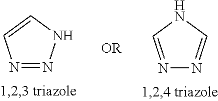 [SMILES: c1c[nH]nn1 ; c1nnc[nH]1]

The term “oxo,” as used herein, refers to a ═O moiety. The term “oxy,” as used herein, refers to a —O— moiety.

“Nitro” refers to the organic compound functional group —NO₂.

“Carbonyl” is a functional group having a carbon atom double-bonded to an oxygen atom (—C═O). “Carboxy” as used herein refers to a —COOH functional group, also written as —(C═O)—OH.

“Amino acid sidechain” as used herein refers to any of the 20 commonly known groups associated with naturally-occurring amino acids, or any natural or synthetic homologue thereof. An “amino acid” includes the sidechain group and the amino group, alpha-carbon atom, and carboxy groups, as commonly described in the art. Examples of amino acids include glycine, and glycine that is substituted with a suitable substituent as described herein, such as alkyl, alkenyl, alkynyl, cycloalkyl, heterocyclo, aryl, heteroaryl, alkoxy, carbonyl, carboxy, etc., or a pharmaceutically acceptable salt or prodrug thereof. For example, “Histidine” is one of the 20 most commonly known amino acids found naturally in proteins. It contains an imidazole side chain substituent. Other examples of naturally-occurring amino acids include lysine, arginine, aspartic acid, glutamic acid, asparagine, glutamine, serine, threonine, tyrosine, alanine, valine, leucine, isoleucine, phenylalanine, methionine, cryptophan, and cysteine. Also included in the definitions of “amino acid sidechain” and “amino acid” is proline, which is commonly included in the definition of an amino acid, but is technically an imino acid. As used in this application, both the naturally-occurring L-, and the non-natural D-amino acid enantiomers are included. A “peptide” is a linear chain of amino acids covalently linked together, typically through an amide linkage, and contains from 1 or 2 to 10 or 20 or more amino acids, and is also optionally substituted and/or branched.

“Boc” or “BOC” is t-butoxycarbonyl, a commonly-known amino protecting group.

A “pharmaceutically acceptable salt” is intended to mean a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

A “prodrug” is intended to mean a compound that is converted under physiological conditions or by solvolysis or metabolically to a specified compound that is pharmaceutically active. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein in their entirety.

Imidazole derivative compounds according to some embodiments include compounds of Formula (X)(I)(a):

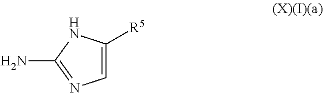

wherein R⁵ is an alkyl, alkenyl or alkynyl having an amide group substituted thereon;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

According to some embodiments, provided are compounds of Formula (X)(I)(a)(1):

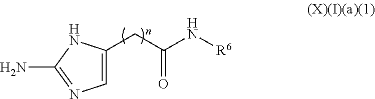

wherein:

n is 1 to 10 carbons, saturated or unsaturated; and

R⁶ is selected from the group consisting of H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments, n is 2, 3, or 4 carbons. In some embodiments, R⁶ is C8 to C20 alkyl (e.g., C8, C9, C10, C11, C12, C13, C14, C15, C15, C16, C17, C18, C19, or C20).

According to some embodiments, provided are compounds of Formula (X)(I)(a)(2):

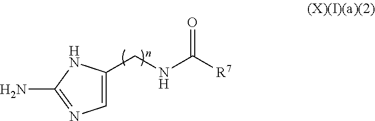

wherein:

n is 1 to 10 carbons, saturated or unsaturated, substituted or unsubstituted; and

R⁷ is selected from the group consisting of H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

According to some embodiments, provided are compounds of Formula (X)(I)(a)(2)(A):

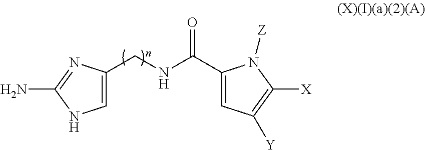

wherein:

n is 1 to 10 carbons, saturated or unsaturated, substituted or unsubstituted; and X, Y and Z are each independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

Also provided are compounds of Formula (I):

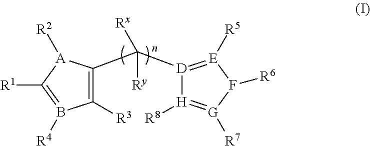

wherein:

each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and

A, B, D, E, F, G and H are each independently selected from carbon, N, S and O, wherein at least one of D, E, F, G and H is carbon; and

n=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (I), R¹ is a substituted amino, A, B, F, G and H are each N, and D and E are each carbon, generally depicted by Formulas (I)(a)(1)-(I)(a)(2):

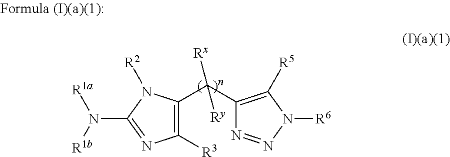

wherein:

R^{1a}, R^{1b}, R², R³, R⁵ and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide; and

each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and

n=0 to 20;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

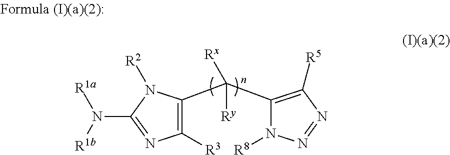

wherein:

R^{1a}, R^{1b}, R², R³, R⁵ and R⁸ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide; and

each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and

n=0 to 20;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (I), R¹ is a substituted amino, A, B, F, G and D are each N, and E and H are each carbon, generally depicted by Formula (I)(b)(2):

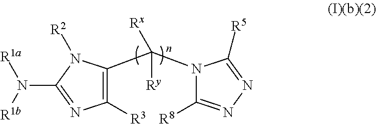

wherein:

R^{1a}, R^{1b}, R², R³, R⁵ and R⁸ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and

n=0 to 20;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

Also provided are compounds of Formula (I)(i):

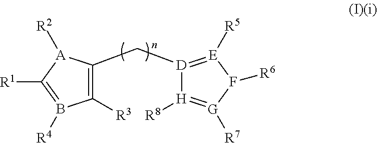

wherein:

each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon; and

n=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (I)(i), R¹ is a substituted amino; R², R³, R⁴, R⁵, R⁷ and R⁸═H; A, B, F, G and H are each N, and D and E are each carbon, generally depicted by Formula (I)(i)(a):

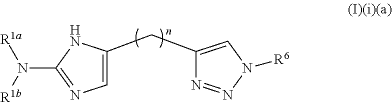

wherein:

R⁶ is selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide; and

n=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

Further provided are compounds of Formula (II):

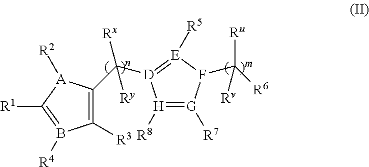

wherein:

each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

each occurrence of R^{x}, R^{y}, R^{u} and R^{v} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon; and

n=0 to 20; and

m=0 to 20;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (II), R¹ is a substituted amino, A, B, F, G and H are each N, and D and E are each carbon, generally depicted by Formula (II)(a):

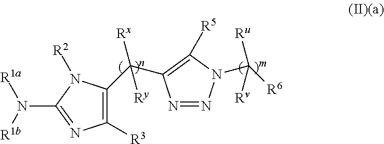

wherein:

R^{1a}, R^{1b}, R², R³, R⁵ and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

each occurrence of R^{x}, R^{y}, R^{u} and R^{v} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

n=0 to 20; and

m=0 to 20;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (II)(a), R^{1a}, R^{1b}, R², R³ and R⁵ are each H and R⁶ is phenyl, e.g., Formula (II)(a)(5)(D), wherein n=5; m=3, and R^{u}=methyl at C2:

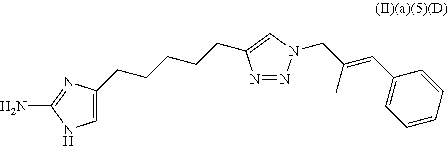 [SMILES: C/C(=C\c1ccccc1)Cn1cc(CCCCCc2c[nH]c(N)n2)nn1]

Also provided are compounds of Formula (II)(i):

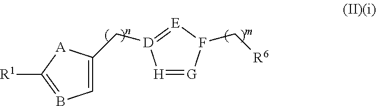

wherein:

R¹ and R⁶ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon;

n=0 to 20, saturated or unsaturated; and

m=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (II)(i), R¹ is a substituted amino, A, B, F, G and H are each N, and D and E are each carbon, generally depicted by Formula (II)(i)(a):

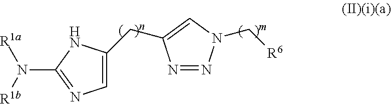

wherein:

R^{1a}, R^{1b} and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

n=0 to 20, saturated or unsaturated; and

m=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (II)(i)(a), R^{1a} and R^{1b} are each H, and R⁶ is heteroaryl.

Also provided are compounds of Formula (IV):

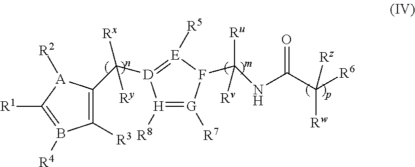

wherein:

R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon; and

n=0 to 20;

m=0 to 20; and

p=0 to 20

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (IV), R¹ is a substituted amino, A, B, F, G and H are each N, and D and E are each carbon, generally depicted by Formula (IV)(a):

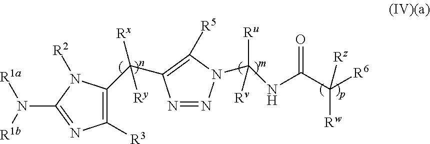

wherein:

R^{1a}, R^{1b}, R², R³, R⁵ and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

n=0 to 20;

m=0 to 20; and

p=0 to 20

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments, R^{1a} and R^{1b} are each H. In some embodiments, R^{1a} and R^{1b} are each H, n=5, m=2, and/or p=0.

In some embodiments, R⁶ is a group:

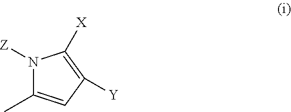

wherein:

X, Y and Z are each independently selected from the group consisting of: H, methyl, Br and Cl.

In some embodiments, R⁶ is a group:

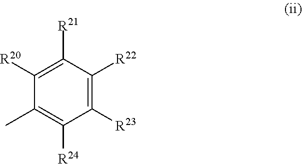

wherein:

R²⁰, R²¹, R²², R²³ and R²⁴ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

Further provided are compounds of Formula (IV)(i):

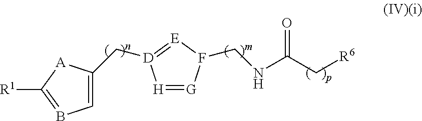

wherein:

R¹ and R⁶ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon;

n=0 to 20, saturated or unsaturated;

m=0 to 20, saturated or unsaturated; and

p=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (IV)(i), R¹ is a substituted amino, A, B, F, G and H are each N, and D and E are each carbon, generally depicted by Formula (IV)(i)(a):

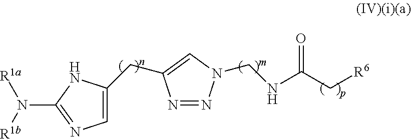

wherein:

R^{1a}, R^{1b} and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

n=0 to 20, saturated or unsaturated;

m=0 to 20, saturated or unsaturated; and

p=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (IV)(i)(a), R^{1a}, R^{1b} and R⁶ are each H. In some embodiments of Formula (IV)(i)(a), R^{1a} and R^{1b} are each H, and R⁶ is aryl or heteroaryl.

Also provided are compounds of Formula (V):

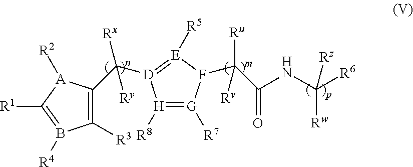

wherein:

R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon; and

n=0 to 20;

m=0 to 20; and

p=0 to 20

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (V), R¹ is a substituted amino, A, B, F, G and H are each N, and D and E are each carbon, generally depicted by Formula (V)(a):

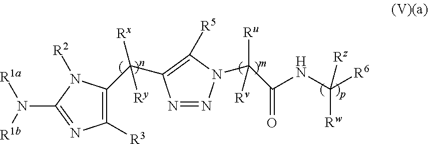

wherein:

R^{1a}, R^{1b}, R², R³, R⁵ and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

n=0 to 20;

m=0 to 20; and

p=0 to 20

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

Further provided are compounds of Formula (V)(i):

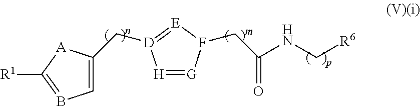

wherein:

R¹ and R⁶ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon;

n=0 to 20, saturated or unsaturated;

m=0 to 20, saturated or unsaturated; and

p=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (V)(i), R¹ is a substituted amino, A, B, F, G and H are each N, and D and E are each carbon, generally depicted by Formula (V)(i)(a):

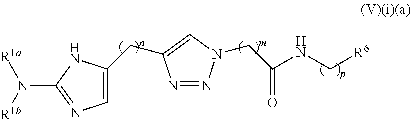

wherein:

R^{1a}, R^{1b} and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

n=0 to 20, saturated or unsaturated;

m=0 to 20, saturated or unsaturated; and

p=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (V)(i)(a), R^{1a}, R^{1b} and R⁶ are each H, alkyl, cycloalkyl or heterocyclo. In some embodiments of Formula (V)(i)(a), R^{1a} and R^{1b} are each H, and R⁶ is aryl.

Also provided are compounds of Formula (VI):

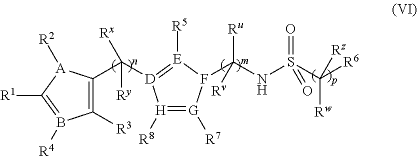

wherein:

R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon; and

n=0 to 20;

m=0 to 20; and

p=0 to 20;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (VI), R¹ is a substituted amino, A, B, F, G and H are each N, and D and E are each carbon, generally depicted by Formula (VI)(a):

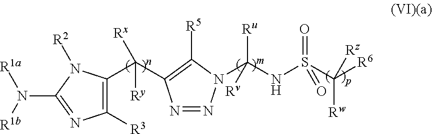

wherein:

R^{1a}, R^{1b}, R², R³, R⁵ and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

n=0 to 20;

m=0 to 20; and

p=0 to 20;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

Further provided are compounds of Formula (VI)(i):

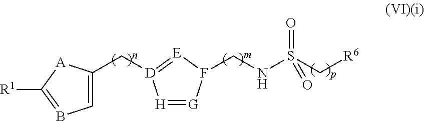

wherein:

R¹ and R⁶ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;

A, B, D, E, F, G and H are each independently selected from carbon, N, S and O, wherein at least one of D, E, F, G and H is carbon;

n=0 to 20, saturated or unsaturated;

m=0 to 20, saturated or unsaturated; and

p=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (VI)(i), R¹ is a substituted amino, A, B, F, G and H are each N, and D and E are each carbon, generally depicted by Formula (VI)(i)(a):

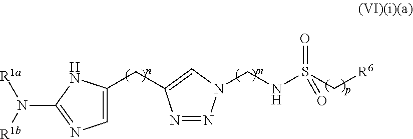

wherein:

R^{1a}, R^{1b} and R⁶ are each independently selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide;

n=0 to 20, saturated or unsaturated;

m=0 to 20, saturated or unsaturated; and

p=0 to 20, saturated or unsaturated;

or a pharmaceutically acceptable salt or prodrug thereof.

This formula may be optionally substituted (e.g., from 1 to 3 or 4 times) with independently selected H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

In some embodiments of Formula (VI)(i)(a), R^{1a} and R^{1b} are each H, and R⁶ is aryl or heteroaryl.

The compounds, compositions and methods of the present disclosure may in particular implementations be constituted as comprising, consisting, or consisting essentially of, some or all of such features, aspects and embodiments, and various elements, ingredients, components, steps, and conditions may be further aggregated in whole or part to constitute various further implementations of the disclosure. For example, the compositions include those comprising, consisting of, or consisting essentially of (e.g., 50%, 60%, 70%, 80%, 90%, 95%, or 99% or more of the total weight or volume of the composition), a component such as response regulator and/or agent such as an imidazole derivative as provided herein.

In some embodiments, kits are provided having: (a) an isolated, purified or substantially purified response regulator or portion thereof, and (b) an agent, e.g., a compound having an imidazole moiety, such as a compound of Formula (X)(I)(a), Formula (I), Formula (I)(a)(1), Formula (II), Formula (IV), or Formula (VI), or a pharmaceutically acceptable salt or prodrug thereof, (c) a container, and optionally, (d) instructions for use. In some embodiments, kits may be used to test a sample containing a biofilm-forming organism (known or unknown) or lysate thereof for the presence of a response regulator that binds to an agent as provided herein, with the isolated, purified or substantially purified response regulator or portion thereof provided as a positive control. The presence of binding of the agent to a response regulator in the biofilm-forming organism or lysate thereof may indicate efficacy in the inhibition or dispersion of the biofilm of the biofilm-forming organism.

Some aspects of the present disclosure are explained in greater detail in the following non-limiting Examples.

### EXPERIMENTAL

### Example 1

It has been demonstrated that imidazole derivative compounds have potent anti-biofilm activity. Herein is demonstrated that biotinylated derivatives of our imidazole-based anti-biofilm agents specifically bind to BfmR, the sensor protein of a two-component system from Acinetobacter baumannii (Accession Numbers: Protein: AAX40744 Gene: AY838282 (bases 185-901)). The imidazole derivative used was the reverse amide:

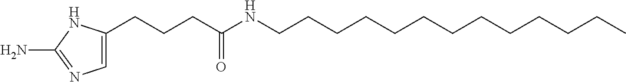 [SMILES: CCCCCCCCCCCCCNC(=O)CCCc1cnc(N)[nH]1]

(See U.S. Patent Application Publication No. 2008/0181923, which is incorporated by reference herein in its entirety). The compound was coupled to biotin as follows.

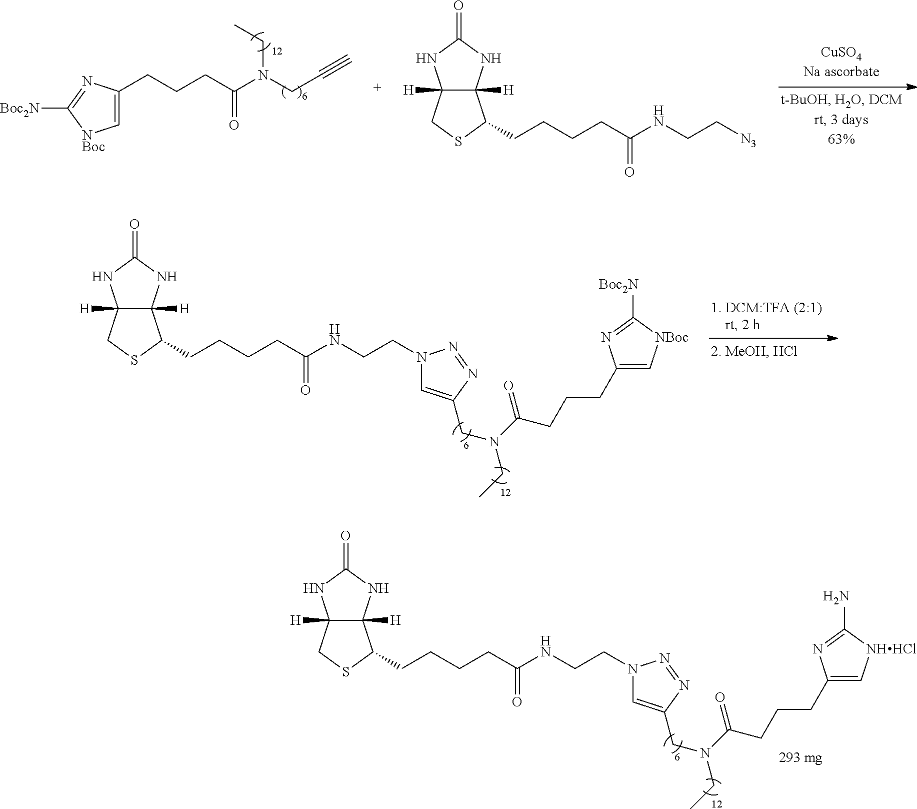

### Experiment:

Magnetic beads coated with streptavidin were washed and resuspended in binding buffer (10 mM Tris, 10 mM KCl, 1 mM EDTA, 1 mM DTT, 0.02% NaN₃ pH 7.5) containing the imidazole derivative compound attached to biotin, a molecule that binds very tightly to the streptavidin on the beads and trapped the compound on the beads. Next, the beads were re-washed to remove any excess compound, washed with blocking buffer (binding buffer containing an appropriate amount of biotin to block unbound streptavidin) and incubated for 5 minutes, washed three times, and then resuspended in A. baumannii lysate, a solution of pure BfmR, or a control solution, and incubated for 30 to 45 minutes at room temperature.

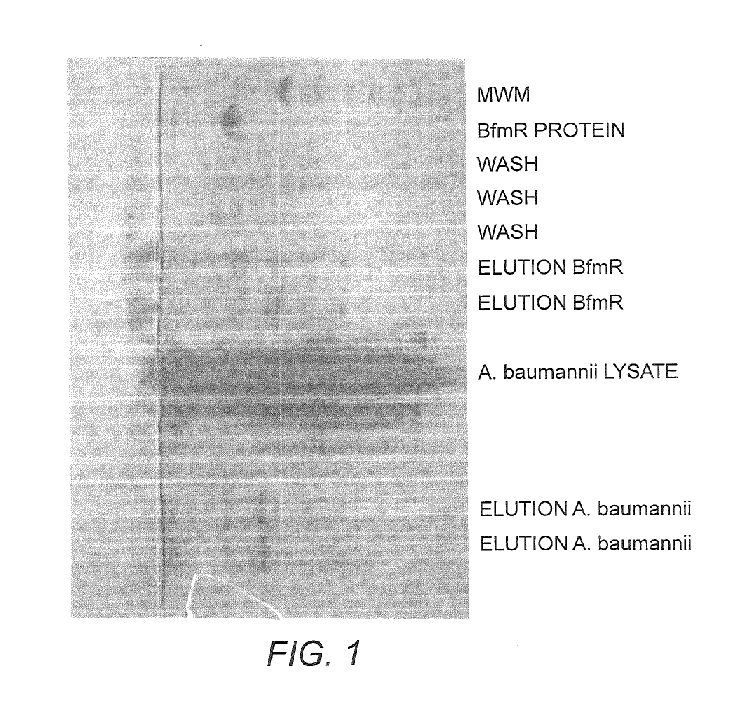

Following the incubation, the beads were thoroughly washed with buffer to remove any excess/non-bound proteins or other cellular materials. Finally, the compound-bound proteins were eluted off of the beads using a low pH buffer (10 mM Tris, 10 mM KCl, 1 mM EDTA, 1 mM DTT, 0.02% NaN₃ pH 2.5), and these elutions were visualized using SDS-PAGE gel electrophoresis. Results are shown in FIG. 1.

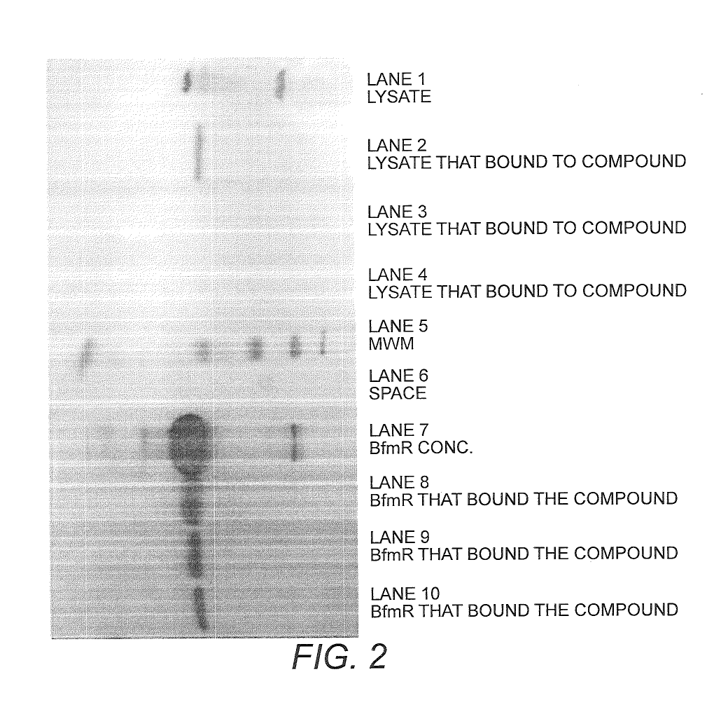

In order to confirm the identity of the protein as A. baumannii BfmR, a western blot was performed using polyclonal antibodies specific to BfmR (kindly provided by Dr. Actis of Miami University, Oxford, Ohio). Results are shown in FIG. 2.

Given the anti-biofilm effect of the imidazole derivative compounds, the observation of direct binding to the Response Regulator BfmR, and previous genetic studies establishing that BfmR is needed for A. baumannii biofilm formation and morphology (see, e.g., Tomaras et al., “Characterization of a two-component regulator system from Acinetobacter baumannii that controls biofilm formation and cellular morphology,” Microbiology (2008), 154, 3398-3409), it is thought that the imidazole derivative compounds may bind to various Response Regulators in their modulation of biofilms.

### Example 2

Computational docking was performed on a structural model of BfmR, the response regulator responsible for biofilm formation in A. baumannii, with the imidazole derivative compound from Example 1:

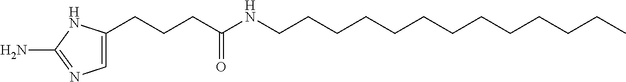 [SMILES: CCCCCCCCCCCCCNC(=O)CCCc1cnc(N)[nH]1]

Computational docking was also performed on a structural model of a different but homologous response regulator protein, GacA, the response regulator responsible for biofilm formation from P. aeuriginosa, with the imidazole derivative compound:

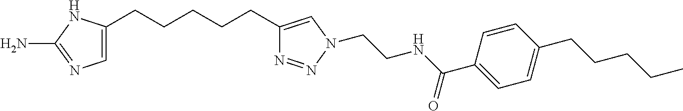 [SMILES: CCCCCc1ccc(C(=O)NCCn2cc(CCCCCc3cnc(N)[nH]3)nn2)cc1]

(See U.S. Patent Application Publication No. 2009/0263438, which is incorporated by reference herein in its entirety).

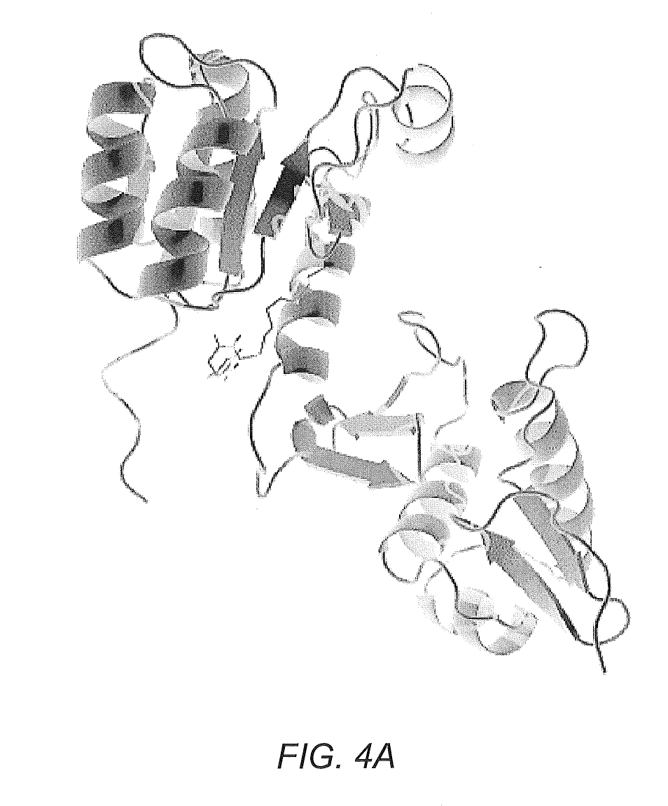

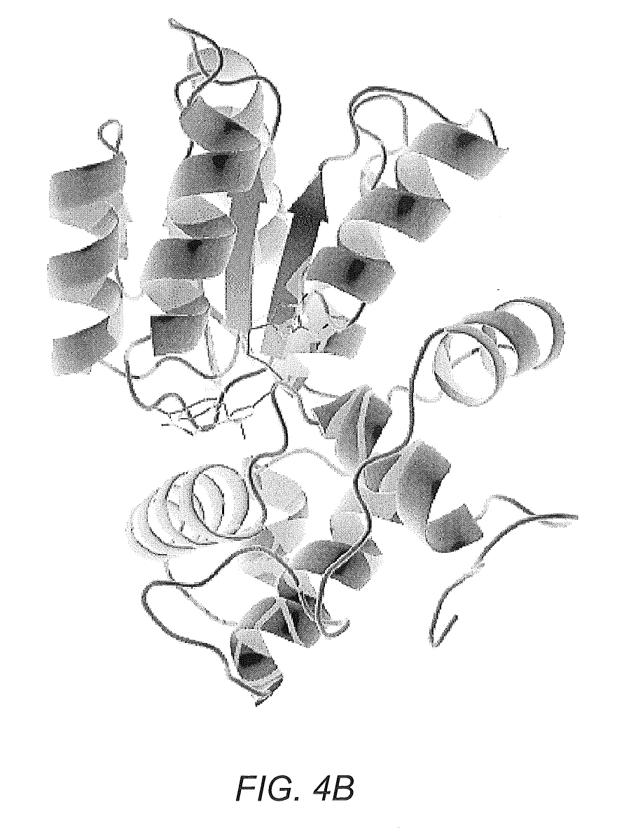

Results are shown in FIGS. 4A-4B. In both cases, the compounds target the N-terminal regulatory domain of the response regulator, in approximately the same place. In both cases, the compounds target the N-terminal regulatory domain of the response regulator, in approximately the same place. These are the lowest energy complexes from the cluster. They do not gravitate towards the DNA-binding C-terminal domain. Thus, the computational work suggests that the N-terminal domain is preferentially targeted.

Without wishing to be bound by theory, two modes of action are possible: one where the compound binds and allosterically inhibits the protein from being phosphorylated/activated, and one where the protein gets phosphorylated but the compound blocks the allosteric communication from the N-terminal domain to the DNA-binding domain.

The foregoing is illustrative of the present disclosure, and is not to be taken as limiting thereof. The invention is defined by the claims, with equivalents of the claims to be included therein.

1. A method of screening:
a compound of Formula (X)(I)(a): 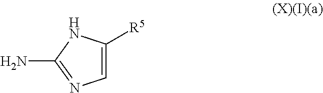
wherein:
R⁵ is an alkyl, alkenyl or alkynyl having an amide group substituted thereon;
or a pharmaceutically acceptable salt or prodrug thereof,
a compound of Formula (I): 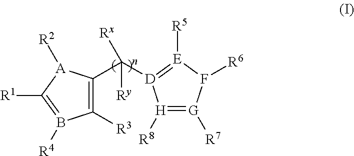
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and
A, B, D, E, F, G and H are each independently selected from carbon, N, S and O, wherein at least one of D, E, F, G and H is carbon; and
n=0 to 20, saturated or unsaturated;
or a pharmaceutically acceptable salt or prodrug thereof,
a compound of Formula (I)(a)(1): 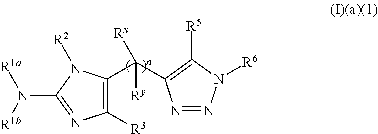
wherein:
R^{1a}, R^{1b}, R², R³, and R⁵ are each independently selected from H and alkyl;
R⁶ is selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide; and
each occurrence of R^{x} and R^{y} is present or absent (depending upon chain saturation), and is independently selected from H and alkyl; and
n=0 to 20;
or a pharmaceutically acceptable salt or prodrug thereof,
a compound of Formula (II): 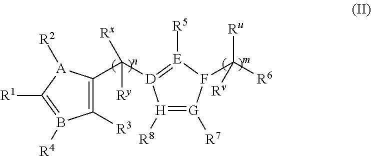
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u} and R^{v} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20; and
m=0 to 20;
or a pharmaceutically acceptable salt or prodrug thereof,
a compound of Formula (IV): 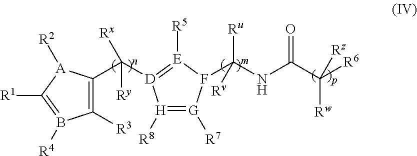
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof,
a compound of Formula (V): 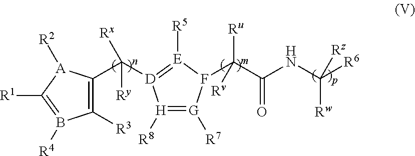
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{y}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20
or a pharmaceutically acceptable salt or prodrug thereof,
or
a compound of Formula (VI): 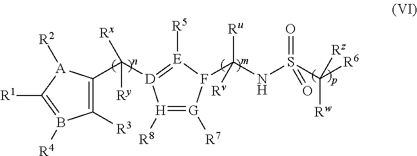
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof, for inhibition of biofilm formation, modulation of microorganism growth, or lowering the minimum inhibitory concentration (MIC) of an antibiotic, said method comprising:
(a) contacting said compound to a response regulator protein or a portion thereof, and then
(b) detecting the presence or absence of binding of said compound to said response regulator protein or portion thereof, said binding indicating said compound has activity in inhibition of biofilm formation, modulation of microorganism growth or lowering the minimum inhibitory concentration (MIC) of an antibiotic.

2. The method of claim 1, wherein said response regulator is an OmpR, NarL or NtrC protein.

3. The method of claim 1, wherein said response regulator is a BfmR, GacA, LytTR, AraC, Spo0A, F is, YcbB, RpoE, MerR, GGDEF, EAL, HD-GYP, CheB, CheC, PP2C, HisK, ANTAR, CsrA, PAS, GAF, TPR, CAP_ED, HPt, PhoB, or CheY protein.

4. The method of claim 1, wherein said response regulator is an OmpR, BfmR or GacA protein.

5. The method of claim 1, wherein said response regulator protein or a portion thereof comprises the N-terminal domain of said response regulator protein.

6. The method of claim 1, wherein said contacting step is carried out in vitro.

7. The method of claim 1, wherein said compound is a compound of Formula (X)(I)(a)(1): 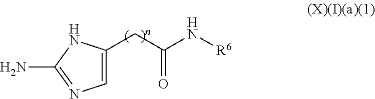
wherein:
n is 2, 3 or 4, saturated or unsaturated; and
R⁶ is selected from the group consisting of H, alkyl, alkenyl and alkynyl;
or a pharmaceutically acceptable salt thereof.

8. The method of claim 7, wherein R⁶ is an alkyl having from 5 to 20 carbon atoms.

9. The method of claim 7, wherein n is 3.

10. The method of claim 1, wherein said compound is coupled to a detectable group.

11. The method of claim 1, wherein said compound is a compound of Formula (IV)(a): 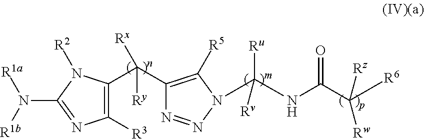
wherein:
R^{1a}, R^{1b}, R², R³, and R⁵ are each H;
each occurrence of R^{x}, R^{y}, R^{u}, R^{v}, R^{z} and R^{w} is present or absent (depending upon chain saturation), and is independently selected from the group consisting of: H and alkyl;
n=3 to 7, saturated or unsaturated;
m=0 to 5, saturated or unsaturated;
p=0 to 5, saturated or unsaturated; and
R⁶ is a group: 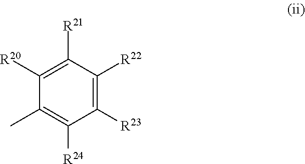
wherein:
R²⁰, R²¹, R²², R²³ and R²⁴ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.
or a pharmaceutically acceptable salt thereof.

12. The method of claim 11, wherein n=5.

13. The method of claim 11, wherein m=2.

14. The method of claim 11, wherein p=0.

15. A method of selecting a compound of Formula (X)(I)(a): 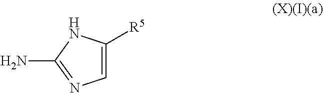
wherein:
R⁵ is an alkyl, alkenyl or alkynyl having an amide group substituted thereon;
or a pharmaceutically acceptable salt or prodrug thereof,
a compound of Formula (I): 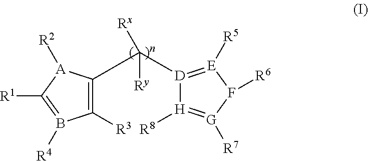
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and
A, B, D, E, F, G and H are each independently selected from carbon, N, S and O, wherein at least one of D, E, F, G and H is carbon; and
n=0 to 20, saturated or unsaturated;
or a pharmaceutically acceptable salt or prodrug thereof,
a compound of Formula (I)(a)(1): 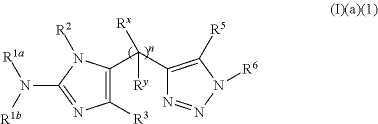
wherein:
R^{1a}, R^{1b}, R², R³, and R⁵ are each independently selected from H and alkyl;
R⁶ is selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide; and
each occurrence of R^{x} and R^{y} is present or absent (depending upon chain saturation), and is independently selected from H and alkyl; and
n=0 to 20;
or a pharmaceutically acceptable salt or prodrug thereof,
a compound of Formula (II): 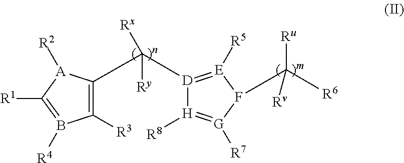
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u} and R^{v} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20; and
m=0 to 20;
or a pharmaceutically acceptable salt or prodrug thereof,
a compound of Formula (IV): 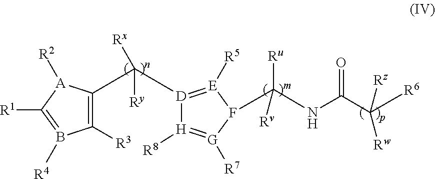
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof,
a compound of Formula (V): 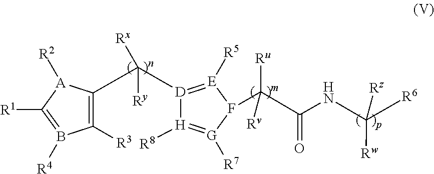
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20
or a pharmaceutically acceptable salt or prodrug thereof,
or
a compound of Formula (VI): 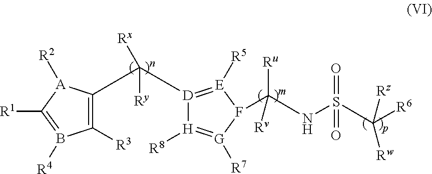
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof,
for activity in treating, reducing or removing a biofilm of interest, modulation of microorganism growth or lowering the minimum inhibitory concentration (MIC) of an antibiotic, comprising:
(a) providing a sample of said biofilm of interest, said sample comprising a response regulator protein or portion thereof,
(b) assaying for binding of said compound to said response regulator protein or portion thereof, said binding indicating said compound has activity in treating, reducing or removing said biofilm of interest, modulating microorganism growth or lowering the minimum inhibitory concentration (MIC) of an antibiotic.

16. The method of claim 15, wherein said sample is a lysate sample.

17. A composition comprising:
(a) an isolated response regulator protein or a portion thereof; and
(b) a compound of Formula (X)(I)(a): 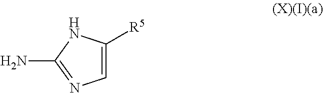
wherein:
R⁵ is an alkyl, alkenyl or alkynyl having an amide group substituted thereon;
or a pharmaceutically acceptable salt or prodrug thereof,
a compound of Formula (I): 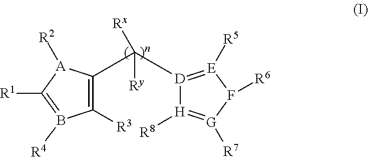
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and
A, B, D, E, F, G and H are each independently selected from carbon, N, S and O, wherein at least one of D, E, F, G and H is carbon; and
n=0 to 20, saturated or unsaturated;
or a pharmaceutically acceptable salt or prodrug thereof,
a compound of Formula (I)(a)(1): 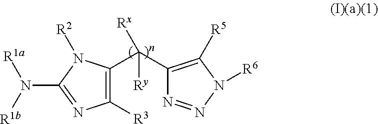
wherein:
R^{1a}, R^{1b}, R², R³, and R⁵ are each independently selected from H and alkyl;
R⁶ is selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide; and
each occurrence of R^{x} and R^{y} is present or absent (depending upon chain saturation), and is independently selected from H and alkyl; and
n=0 to 20;
or a pharmaceutically acceptable salt or prodrug thereof,
a compound of Formula (II): 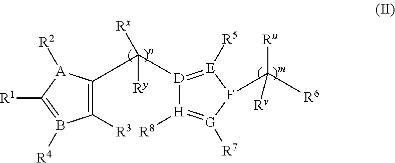
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u} and R^{v} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20; and
m=0 to 20;
or a pharmaceutically acceptable salt or prodrug thereof,
a compound of Formula (IV): 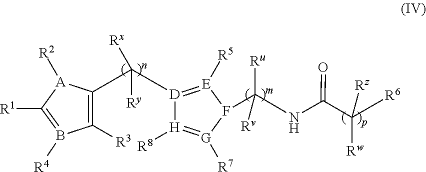
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof,
a compound of Formula (V): 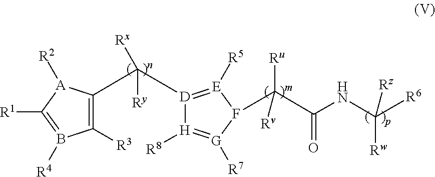
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20
or a pharmaceutically acceptable salt or prodrug thereof,
or
a compound of Formula (VI): 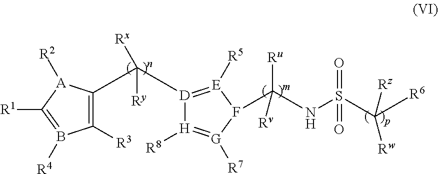
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof.

18. The composition of claim 17, wherein said response regulator is an OmpR, NarL or NtrC protein.

19. The composition of claim 17, wherein said response regulator is a BfmR, GacA, LytTR, AraC, Spo0A, F is, YcbB, RpoE, MerR, GGDEF, EAL, HD-GYP, CheB, CheC, PP2C, HisK, ANTAR, CsrA, PAS, GAF, TPR, CAP_ED, HPt, PhoB, or CheY protein.

20. The composition of claim 17, wherein said response regulator is an OmpR, BfmR or GacA protein.

21. The composition of claim 17, wherein said composition is an aqueous composition.

22. A kit comprising the composition of claim 17, a container, and optionally instructions for use.

23. A compound of Formula (X)(I)(a): 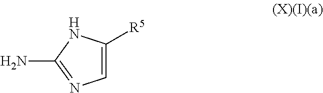
wherein:
R⁵ is an alkyl, alkenyl or alkynyl having an amide group substituted thereon;
or a pharmaceutically acceptable salt or prodrug thereof,
a compound of Formula (I): 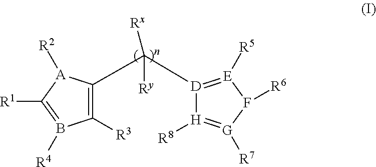
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and
A, B, D, E, F, G and H are each independently selected from carbon, N, S and O, wherein at least one of D, E, F, G and H is carbon; and
n=0 to 20, saturated or unsaturated;
or a pharmaceutically acceptable salt or prodrug thereof,
a compound of Formula (I)(a)(1): 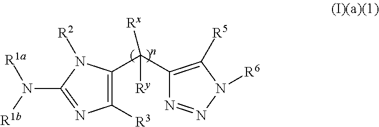
wherein:
R^{1a}, R^{1b}, R², R³, and R⁵ are each independently selected from H and alkyl;
R⁶ is selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide; and
each occurrence of R^{x} and R^{y} is present or absent (depending upon chain saturation), and is independently selected from H and alkyl; and
n=0 to 20;
or a pharmaceutically acceptable salt or prodrug thereof,
a compound of Formula (II): 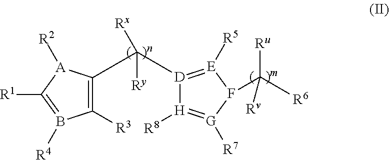
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u} and R^{v} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20; and
m=0 to 20;
or a pharmaceutically acceptable salt or prodrug thereof,
a compound of Formula (IV): 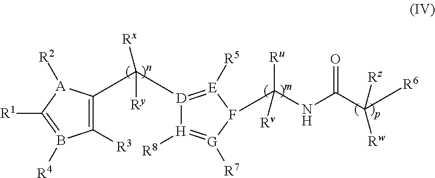
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof,
a compound of Formula (V): 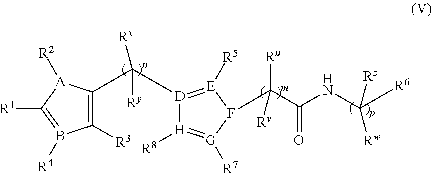
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20
or a pharmaceutically acceptable salt or prodrug thereof,
or
a compound of Formula (VI): 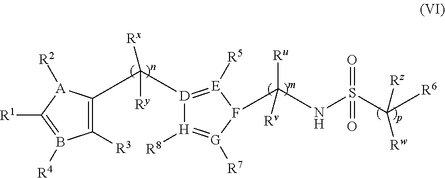
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof,
wherein said compound is coupled to a detectable group.

24. The compound of claim 23, wherein said compound is covalently coupled to said detectable group.

25. The compound of claim 23, wherein said detectable group is a radiolabel, a gold bead, a chemiluminescence label, a ligand (e.g., biotin, digoxin), a fluorescence label (e.g., rhodamine, phycoerythrin, fluorescein), a fluorescent protein (e.g., green fluorescent protein or one of its many modified forms), a nucleic acid, or an energy absorbing/emitting agent.

26. A method of inhibiting biofilm formation of a cellular species capable of such formation, said method comprising binding of a response regulator of said cellular species with an exogenous agent, said response regulator selected from among a OmpR protein and a BfmR protein, and said agent provided in an amount effective to modulate biofilm-mediating communication of said cellular species, to thereby inhibit biofilm formation of said cellular species.

27. The method of claim 26, wherein said cellular response regulator is BfmR protein or GacA protein.

28. A method of modulating biofilm activity of a biofilm-producing cellular species, comprising binding surface bound BfmR of cells of said cellular species with an exogenous agent, said agent provided in an amount effective to at least partially suppress biofilm-mediating BfmR/histidine kinase communication of said cells.

29. A method of disrupting quorum sensing of a cellular species capable of biofilm production, comprising binding surface bound BfmR of cells of said cellular species with an exogenous agent, said agent provided in an amount effective to at least partially suppress biofilm-mediating BfmR/histidine kinase communication of said cells.

30. A method of inhibiting antibiotic resistance of a cellular species, comprising binding surface bound BfmR of cells of said cellular species with an exogenous agent, said agent provided in an amount effective to at least partially suppress biofilm-mediating BfmR/histidine kinase communication of said cells, to thereby inhibit antibiotic resistance.

31. The method of claim 26, wherein the exogenous agent comprises a compound having an imidazole moiety.

32. The method of claim 26, wherein said exogenous agent comprises a compound of Formula (X)(I)(a): 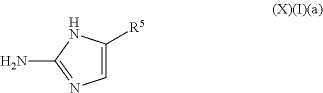
wherein:
R⁵ is an alkyl, alkenyl or alkynyl having an amide group substituted thereon;
or a pharmaceutically acceptable salt or prodrug thereof,
a compound of Formula (I): 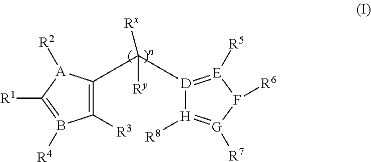
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x} and R^{y} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide; and
A, B, D, E, F, G and H are each independently selected from carbon, N, S and O, wherein at least one of D, E, F, G and H is carbon; and
n=0 to 20, saturated or unsaturated;
or a pharmaceutically acceptable salt or prodrug thereof,
a compound of Formula (I)(a)(1): 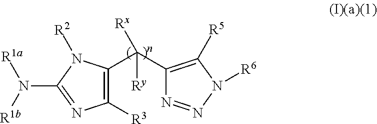
wherein:
R^{1a}, R^{1b}, R², R³, and R⁵ are each independently selected from H and alkyl;
R⁶ is selected from the group consisting of: H, hydroxy, acyl, alkyl, alkenyl, alkynyl, cycloalkyl, heterocyclo, aryl, heteroaryl, alkoxy, amino, amide, sulfone, sulfoxide, oxo, oxy, nitro, carbonyl, carboxy, amino acid sidechain, amino acid and peptide; and
each occurrence of R^{x} and R^{y} is present or absent (depending upon chain saturation), and is independently selected from H and alkyl; and
n=0 to 20;
or a pharmaceutically acceptable salt or prodrug thereof,
a compound of Formula (II): 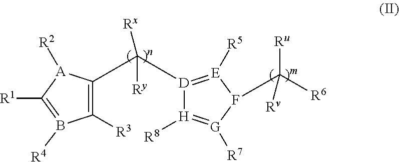
wherein:
each occurrence of R¹, R², R³, R⁴, R⁵, R⁶, R⁷ and R⁸ (depending upon valence requirement) is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u} and R^{v} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20; and
m=0 to 20;
or a pharmaceutically acceptable salt or prodrug thereof,
a compound of Formula (IV): 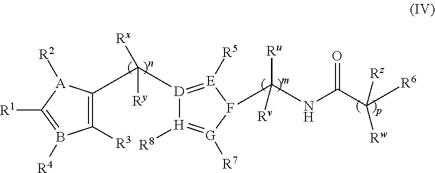
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof,
a compound of Formula (V): 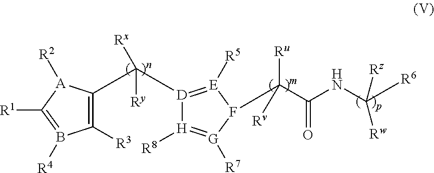
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20
or a pharmaceutically acceptable salt or prodrug thereof,
or
a compound of Formula (VI): 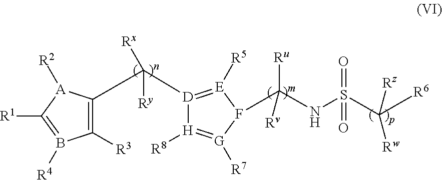
wherein:
R¹, R², R³, R⁴, R⁵, R⁶, R⁷ and R⁸ are each independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
each occurrence of R^{x}, R^{y}, R^{u}, R^{v}, R^{z} and R^{w} is present or absent (depending upon chain saturation), and is independently selected from the group consisting of: H, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide;
A, B, D, E, F, G and H are each independently selected from carbon, N, S and O, wherein at least one of D, E, F, G and H is carbon; and
n=0 to 20;
m=0 to 20; and
p=0 to 20;
or a pharmaceutically acceptable salt or prodrug thereof.

33. The method of claim 26, wherein said cellular species comprises bacteria of the genus Acinetobacter, Pseudomonas, or Vibrio.

34. The method of claim 26, wherein said cellular species comprises A. baumannii or P. aeuriginosa.


SEQUENCE LISTING (5 sequences)

SEQ ID NO: 1 - protein, 238 aa, Acinetobacter baumannii
MSQEEKLPKILIVEDDERLARLTQEYLIRNGLEVGVETDGNRAIRRIISEQPDLVVLDVMLPGADGLTVCREVRPHYHQPILMLTARTEDMDQVLGLEMGADDYVAKPVQPRVLLARIRALLRRTDKTVEDEVAQRIEFDDLVIDNGGRSVTLNGELVDFTSAEYDLLWLLASNAGRILSREDIFERLRGIEYDGQDRSIDVRISRIRPKIGDDPENPKRIKTVRSKGYLFVKETNGL

SEQ ID NO: 2 - protein, 239 aa, Escherichia coli
MQENYKILVVDDDMRLRALLERYLTEQGFQVRSVANAEQMDRLLTRESFHLMVLDLMLPGEDGLSICRRLRSQSNPMPIIMVTAKGEEVDRIVGLEIGADDYIPKPFNPRELLARIRAVLRRQANELPGAPSQEEAVIAFGKFKLNLGTREMFREDEPMPLTSGEFAVLKALVSHPREPLSRDKLMNLARGREYSAMERSIDVQISRLRRMVEEDPAHPRYIQTVWGLGYVFVPDGSKA

SEQ ID NO: 3 - protein, 239 aa, Xanthomonas campestris
MDLQQPCVLVVDDDPDLRKLIGEFLSAHGYQVDMAENVAEMRTAMAQRRPDLIVLDVMMPGEDGLSAARALASERGSPAVIMLSALGNDTDRIIGLEVGADDYLAKPCNPRELLARVRALLRRSQASNEQADQRGNVYEFAGWRLDVVRRDLRDPTGIFINLSDGEFALLRTFVEHPQRVLSRDQLLDYARGRDTDVYDRAIDSQISRLRRKINERVHTELIRTVRNEGYMLLPGVSRL

SEQ ID NO: 4 - protein, 213 aa, Pseudomonas aeruginosa
MIKVLVVDDHDLVRTGITRMLADIEGLQVVGQADCGEDCLKLARELKPDVVLMDVKMPGIGGLEATRKLLRSQPDIKVVVVTVCEEDPFPTRLMQAGAGYMTKGAGLEEMVQAIRQVFAGQRYISPQIAQQLALKSFQPQQHDSPFDSLSEREIQIALMIANCHKVQSISDKLCLSPKTVNTYRYRIFEKLSITSDVELALLAVRHGMVDAAS

SEQ ID NO: 5 - protein, 246 aa, Pseudomonas aeruginosa
MEHVDHILIVDDDREIRELVGNYLKKNGLRTTIVADGRQMRAFLEANTVDLIVLDIMMPGDDGLLLCRELRVGKHKATPVLMLTARNDETDRIIGLEMGADDYLTKPFSARELLARINAVLRRTRMLPPNLTVSESSRLIGFGQWQLDTSARHLLDDAGTVVALSGAEYRLLRVFLDHPQRVLSRDQLLNLTQGREADIFDRSIDLLVSRLRQRLGDDAREPEYIKTVRSEGYVFSLPVRLVEAHP